(19) United States
(12) Patent Application Publication
(10) Pub. No.: US 2024/0148411 A1
(43) Pub. Date: May 9, 2024

(54) UTERINE MANIPULATOR FOR ROBOTIC SURGICAL SYSTEM

(71) Applicant: Cilag GmbH International, Zug (CH)

(72) Inventors: Andrew Jernberg, San Francisco, CA (US); Tracey A. Morley, Sunnyvale, CA (US)

(21) Appl. No.: 17/983,513

(22) Filed: Nov. 9, 2022

(51) Int. Cl. A61B 17/42; A61B 34/00; A61B 34/30

(52) U.S. Cl. CPC A61B 17/4241; A61B 34/30; A61B 34/74; A61B 2017/00477; A61B 2017/4216; A61B 2034/305

(57) ABSTRACT

A robotic surgical system includes a robotic arm, a tool driver, and a surgical instrument. The robotic arm that includes a distal end. The tool driver is operatively coupled with the distal end of the robotic arm. The surgical instrument includes a uterine manipulator and a tool drive adapter. The uterine manipulator is configured to be inserted into a uterus of a patient. The tool drive adapter is configured to move from a non-engaged configuration to an engaged configuration. In the non-engaged configuration, the tool drive adapter is spaced a distance from the uterine manipulator. In the engaged configuration, the uterine manipulator and the tool drive adapter are engaged. The tool drive adapter is operatively coupled with the tool driver in the non-engaged and engaged configurations.

### BACKGROUND

A variety of medical instruments may be used in procedures conducted by a medical professional operator, as well as applications in robotically assisted surgeries. In the case of robotically assisted surgery, the clinician may operate a master controller to remotely control the motion of such medical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletol gloves, master manipulators, or the like), which are coupled by a servo mechanism to the medical instrument. In some scenarios, a servo motor moves a manipulator supporting the medical instrument based on the clinician's manipulation of the hand input devices. During the medical procedure, the clinician may employ, via a robotic system, a variety of medical instruments including an ultrasonic blade, a surgical stapler, a tissue grasper, a needle driver, an electrosurgical cautery probes, etc. Each of these structures performs functions for the clinician, for example, cutting tissue, coagulating tissue, holding or driving a needle, grasping a blood vessel, dissecting tissue, or cauterizing tissue.

Examples of robotic systems are described in U.S. Pat. No. 9,763,741, entitled “System for Robotic-Assisted Endolumenal Surgery and Related Methods,” issued Sep. 19, 2017, the disclosure of which is incorporated by reference herein, in its entirety; U.S. Pat. No. 10,464,209, entitled “Robotic System with Indication of Boundary for Robotic Arm,” issued Nov. 5, 2019, the disclosure of which is incorporated by reference herein, in its entirety; U.S. Pat. No. 10,667,875, entitled “Systems and Techniques for Providing Multiple Perspectives During Medical Procedures,” issued Jun. 2, 2020, the disclosure of which is incorporated by reference herein, in its entirety; U.S. Pat. No. 10,765,303, entitled “System and Method for Driving Medical Instrument,” issued Sep. 8, 2020, the disclosure of which is incorporated by reference herein, in its entirety; U.S. Pat. No. 10,827,913, entitled “Systems and Methods for Displaying Estimated Location of Instrument,” issued Nov. 10, 2020, the disclosure of which is incorporated by reference herein, in its entirety; U.S. Pat. No. 10,881,280, entitled “Manually and Robotically Controllable Medical Instruments,” issued Jan. 5, 2021, the disclosure of which is incorporated by reference herein, in its entirety; U.S. Pat. No. 10,898,277, entitled “Systems and Methods for Registration of Location Sensors,” issued Jan. 26, 2012, the disclosure of which is incorporated by reference herein, in its entirety; and U.S. Pat. No. 11,058,493, entitled “Robotic System Configured for Navigation Path Tracing,” issued Jul. 13, 2021, the disclosure of which is incorporated by reference herein, in its entirety.

During a hysterectomy procedure, a colpotomy may be performed at the cervicovaginal junction. Such procedures may include the use of a uterine manipulator that includes a colpotomy cup or similar structure. Examples of instruments that may be used during a hysterectomy procedure are described in U.S. Pat. No. 9,743,955, entitled “Intracorporeal Transilluminator of Tissue Using LED Array,” issued Aug. 29, 2017; U.S. Pat. No. 9,788,859, entitled “Uterine Manipulators and Related Components and Methods,” issued Oct. 17, 2017; U.S. Pat. No. 10,639,072, entitled “Uterine Manipulator,” issued May 5, 2020; U.S. Pub. No. 2021/0100584, entitled “Uterine Manipulator,” published Apr. 8, 2021; U.S. Pub. No. 2018/0325552, entitled “Colpotomy Systems, Devices, and Methods with Rotational Cutting,” published Nov. 15, 2018.

While several medical instruments, systems, and methods have been made and used, it is believed that no one prior to the inventors has made or used the invention described in the appended claims.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 depicts a perspective view of an example of a table-based robotic system that includes a control console and a plurality of robotic arms;

FIG. 2 depicts a perspective view of an example of a robotic arm, an example of a tool drive, and an example of a surgical instrument, each configured with use with the table-based robotic system of FIG. 1;

FIG. 3A depicts an enlarged schematic perspective view of the tool driver and surgical instrument of FIG. 2;

FIG. 3B depicts an schematic perspective view of the tool driver of FIG. 3A, but with the surgical instrument removed to expose rotary drives;

FIG. 4A depicts a perspective view of an example of a robotic surgical system that includes a portion of another example of a robotic arm, another example of a tool driver, and an example of a surgical instrument that includes a uterine manipulator and a tool drive adapter;

FIG. 4B depicts a perspective view of the robotic surgical system of FIG. 4A, but with the uterine manipulator coupled with the tool drive adapter, with a carriage of the tool driver moved distally;

FIG. 5 depicts an enlarged perspective view of a portion of FIG. 4B;

FIG. 6 depicts a front view of a coupling feature of the tool driver of FIG. 4A;

FIG. 7 depicts a side view of the uterine manipulator of FIG. 4A;

FIG. 8 depicts an enlarged perspective view of a coupling feature of the uterine manipulator of FIG. 7;

FIG. 9 depicts a partial cross-sectional view of components shown in FIG. 4B, taken along line 9-9 of FIG. 4B prior to a disengagement feature being actuated;

FIG. 10A depicts a perspective view of a clinician inserting the uterine manipulator of FIG. 4A into the vagina of a patient;

FIG. 10B depicts a perspective view of a clinician coupling the uterine manipulator of FIG. 4A with the tool driver of FIG. 10A while the uterine manipulator is disposed in the vagina of the patient;

FIG. 10C depicts a perspective view of a clinician translating a carriage of the tool driver of FIG. 10B to couple the uterine manipulator of FIG. 4A with the tool driver adapter of FIG. 4A while the uterine manipulator is disposed in the vagina of the patient;

FIG. 11A depicts a schematic perspective view of another example of a tool drive adapter coupled with a carriage of another tool driver, where the tool drive adapter includes a handle and a cartridge;

FIG. 11B depicts a schematic perspective view of coupling features of the handle of FIG. 11A being actuated to decouple the handle from the cartridge;

FIG. 11C depicts a schematic perspective view of the handle of FIG. 11B being removed to provide access to a coupling feature of the cartridge;

FIG. 11D depicts a schematic perspective view of the coupling feature of the cartridge of FIG. 11C being actuated;

FIG. 11E depicts a schematic perspective view of the cartridge of FIG. 11D being removed to reveal outputs of the carriage of FIG. 11A;

FIG. 12A depicts a schematic perspective view of another example of a tool drive adapter coupled with a carriage of a tool driver, where the tool drive adapter includes a handle and a cartridge;

FIG. 12B depicts an enlarged schematic perspective view of a coupling feature of the handle of FIG. 12A actuated to decouple the handle from the cartridge;

FIG. 12C depicts a schematic perspective view of the handle of FIG. 12B being removed while the cartridge remains coupled with the carriage according to a first decoupling sequence;

FIG. 12D depicts a schematic perspective view of coupling features of the cartridge of FIG. 12A being actuated to decouple the cartridge from the carriage; and

FIG. 12E depicts a schematic perspective view of the handle and cartridge of FIG. 12A being collectively removed from the carriage according to a second decoupling sequence.

### DETAILED DESCRIPTION

### I. Overview of Example of Robotic Surgical System

Aspects of the present disclosure may be integrated into a robotically-enabled medical system capable of performing a variety of medical procedures, including both minimally invasive, such as laparoscopy, and non-invasive, such as endoscopy, procedures. Among endoscopy procedures, the system may be capable of performing bronchoscopy, ureteroscopy, gastroscopy, etc.

In addition to performing the breadth of procedures, the system may provide additional benefits, such as enhanced imaging and guidance to assist the clinician. Additionally, the system may provide the clinician with the ability to perform the procedure from an ergonomic position without the need for awkward arm motions and positions. Still further, the system may provide the clinician with the ability to perform the procedure with improved ease of use such that one or more of the instruments of the system can be controlled by a single user.

Various embodiments will be described below in conjunction with the drawings for purposes of illustration. It should be appreciated that many other implementations of the disclosed concepts are possible, and various advantages can be achieved with the disclosed implementations. Headings are included herein for reference and to aid in locating various sections. These headings are not intended to limit the scope of the concepts described with respect thereto. Such concepts may have applicability throughout the entire specification.

A. Example of Robotic System Table

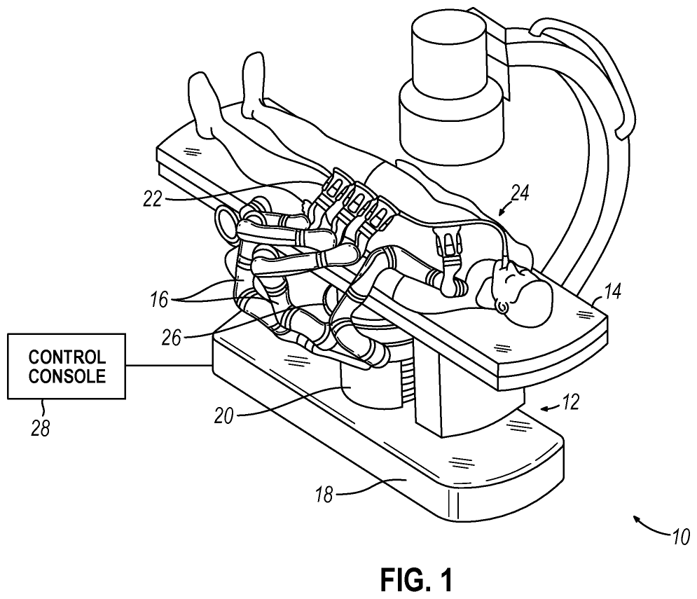

FIG. 1 illustrates an example of a robotic surgical system (10). Robotic surgical system (10) includes a support structure (12) for supporting a platform (14) (shown as a “table” or “bed”) over the floor and one or more robotic arms (16). Support structure (12) includes a base (18) and a column (20). Column (20) structurally supports platform (14), and provides a path for vertical translation of the carriages. In some versions, a table base may stow and store robotic arms (16) when not in use. Column (20) of the present example also includes a ring-shaped carriage (26), from which robotic arms (16) are based. A control console (28) is coupled with robotic surgical system (10).

Robotic arms (16) are shown as part of a table-mounted system, but in other configurations, robotic arms (16) may be mounted in a cart, ceiling or sidewall, or other suitable support surface. Robotic arms (16) are shown as extending from column (20) via carriage (26). However, robotic arms (16) may be coupled with robotic surgical system (10) using a variety of suitable structures. While robotic arms (16) are all shown as being positioned on one side of the patient in FIG. 1, other configurations may position robotic arms (16) on both sides of the patient, between the legs of the patient, and/or in any other suitable locations. Tool drivers (22) are positioned at distal ends of robotic arms (16) in the present example. Tool drivers (22) are operable to manipulate one or more surgical instruments (24), as will be described in greater detail below.

B. Example of a Robotic Arm, Tool Drive, and Tool

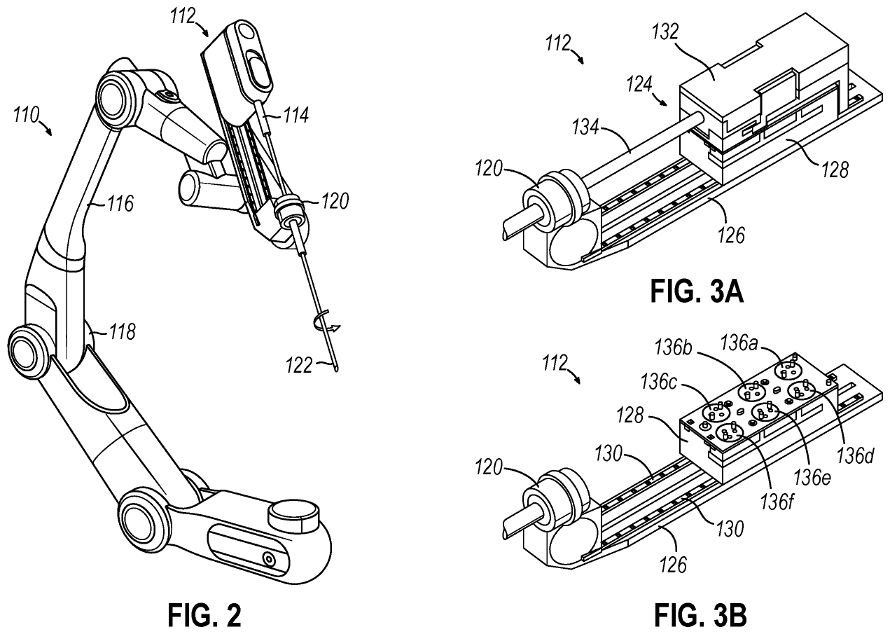

FIG. 2 shows an example of a robotic arm (110), a tool driver (112), and a surgical instrument (114), which may be incorporated into robotic surgical system (10) in place of a robotic arm (16), a tool driver (22), and a surgical instrument (24) that are shown in FIG. 1. Additional examples of robotic arms, a tool drivers, and a surgical instruments are shown and described in U.S. Pat. No. 10,166,082, entitled “System and Method for Controlling a Robotic Wrist,” issued Jan. 1, 2019, the disclosure of which is incorporated by reference herein, in its entirety.

As shown in FIG. 2, robotic arm (110) includes a plurality of links (116) and a plurality of joints (118) for actuating links (116) relative to one another. Tool driver (112) is attached to the distal end of robotic arm (110). Tool driver (112) includes a cannula (120) coupled to the end of tool driver (112), to receive and guide surgical instrument (114). Surgical instrument (114) may include an endoscope, a laparoscope, a stapler, graspers, an ultrasonic instrument, an RF electrosurgical instrument, or any other suitable kind of instrument. Surgical instrument (114) is inserted into the patient via cannula (120). The distal end of surgical instrument (114) includes an end effector (122). End effector (122) is configured to interact with the patient (e.g., providing visualization, stapling, grasping, ultrasonic cutting and/or sealing, electrosurgical cutting and/or sealing, etc.).

Joints (118) of robotic arm (110) may be actuated to selectively position and orient tool driver (112), which actuates the end effector (122) for robotic surgeries. Joints (118) may include various types, such as a pitch joint or a roll joint, which may substantially constrain the movement of the adjacent links (116) around certain axes relative to other links (116). Each joint (118) represents an independent degree of freedom available to robotic arm (110). A multitude of joints (118) result in a multitude of degrees of freedom, allowing for “redundant” degrees of freedom. Redundant degrees of freedom allow the robotic arms (110) to position their respective end effectors (122) at a specific position, orientation, and trajectory in space using different positions links (116) and angles of joints (118). This allows for the system to position and direct a surgical instrument (114) from a desired point in space while allowing the clinician to move joints (118) into a clinically advantageous position away from the patient to create greater access, while avoiding collisions of robotic arms (110).

FIGS. 3A and 3B show tool driver (112) with and without a tool driver adapter (124), which may also be referred to as a tool base. As shown in FIGS. 3A and 3B, tool driver (112) may include a stage (126) and a carriage (128). Stage (126) includes longitudinal tracks (132). Carriage (128) is slidingly engaged with longitudinal tracks (130). Stage (126) may be configured to couple to the distal end of robotic arm (110) such that articulation of robotic arm (110) positions and/or orients tool driver (112) in space. Surgical instrument (114) includes a tool driver adapter (124) at a proximal end and, as noted above, end effector (122) at a distal end. Tool driver adapter (124) includes a handle (132) and a shaft assembly (134) that extends distally from handle (132).

Carriage (128) is configured to couple with tool driver adapter (124). Carriage (128) may drive a set of articulated movements of end effector (122) and/or otherwise actuate end effector (122), such as through a cable system or wires manipulated and controlled by actuated drives. Carriage (128) may include different configurations of actuated drives, including but not limited to motorized rotary axis drives. The plurality of rotary axis drives may be arranged in any suitable manner. As shown in FIG. 3B, carriage (128) of the present example includes six rotary drives (134a-f) arranged in two rows, extending longitudinally along the base of carriage (128). Rotary drives (134a-c) are arranged in a first row that is longitudinally offset from a second row in which rotary drives (134d-f) are arranged. This staggered arrangement of rotary drives (134a-f) may reduce the width of carriage (128) and thereby provide a more compact form factor for tool driver (112). However, rotary drives (134a-f) may be provided in any other suitable arrangement. Moreover, any other suitable kind(s) of drive outputs may be provided by carriage (128), in addition to or in lieu of rotary drives (134a-f).

### II. Example of a Robotic Surgical System

A. Overview

The coupling of a surgical instrument (24, 114) with a tool driver (22, 112) or other feature of robotic arm (16, 110) may be cumbersome in some scenarios. For example, the mass and/or volume of certain surgical instruments (24, 114) may cause their coupling with a tool driver (22, 112) or other feature of robotic arm (16, 110) challenging. This challenge may be increased when a majority of the mass and/or volume is at or near the proximal or distal end of surgical instrument (24, 114), which may cause surgical instrument (24, 114) to feel substantially imbalanced in the hand of the clinician as the clinician tries to couple surgical instrument (24, 112) with a tool driver (22, 112) or other feature of robotic arm (16, 110). This imbalanced nature of surgical instrument (24, 114) may result in reduced control of surgical instrument (24, 114) relative to the patient anatomy, before and during coupling of surgical instrument (24, 114) with a tool driver (22, 112) or other feature of robotic arm (16, 110), particularly if a distal portion of surgical instrument (24, 114) is already disposed in the patient before surgical instrument (24, 114) is coupled with a tool driver (22, 112) or other feature of robotic arm (16, 110). It may be desirable for the clinician to improve patient comfort by providing and maintaining accurate and precise placement of the distal end of surgical instrument (24, 114) within the patient, before and during coupling of surgical instrument (24, 114) with a tool driver (22, 112) or other feature of robotic arm (16, 110). Additionally, it may be desirable for surgical instrument (24, 114) to remain in the position initially placed, without shifting or otherwise moving within the patient, before and during coupling of surgical instrument (24, 114) with a tool driver (22, 112) or other feature of robotic arm (16, 110).

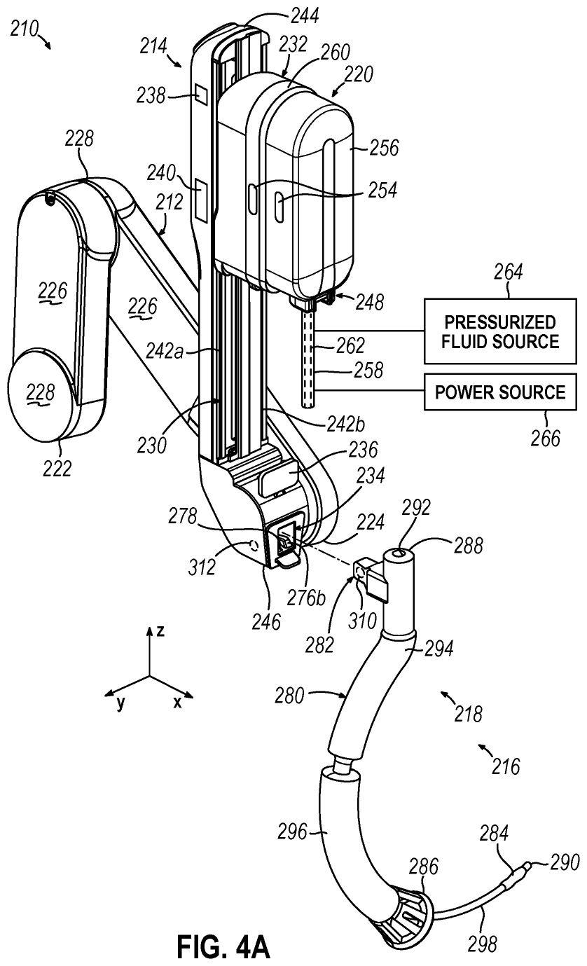

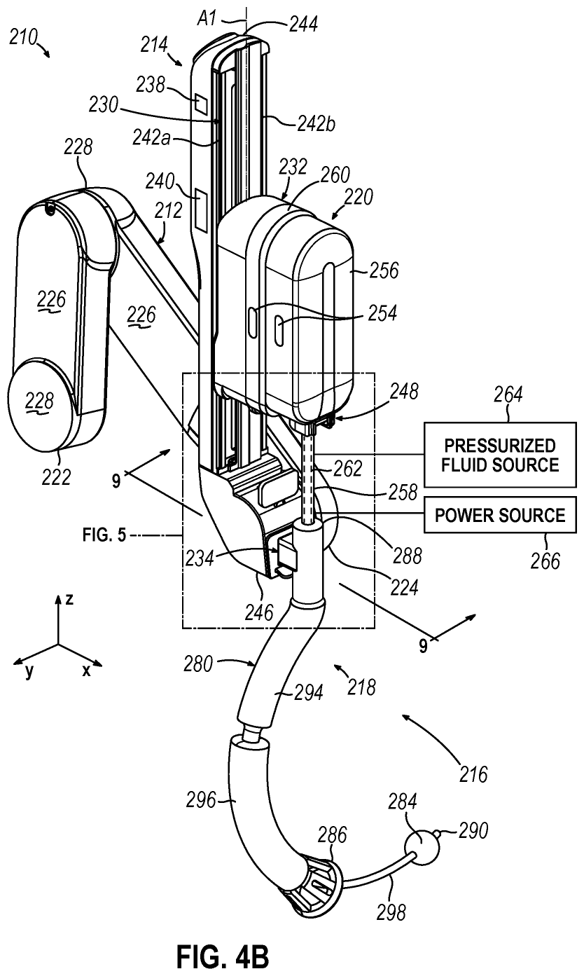

One example of a kind of instrument (24, 114) that may provide difficulties when imbalanced is a uterine manipulator. For instance, some procedures may call for a distal end of the uterine manipulator to be inserted into the patient before the proximal end of the uterine manipulator is coupled with a tool driver (22, 112) or other feature of robotic arm (16, 110). In cases where the proximal end of the uterine manipulator is substantially heavier than the rest of the uterine manipulator, it may be difficult for the clinician to maintain the position and orientation of the distal end of the uterine manipulator in the patient before coupling the proximal end of the uterine manipulator with a tool driver (22, 112) or other feature of robotic arm (16, 110). It may therefore be desirable to provide a variation of a uterine manipulator that does not provide this kind of imbalance and associated difficulties. To that end, FIGS. 4A-4B show an example of a robotic surgical system (210) that includes at least one robotic arm (212), a first example of a tool drive assembly (214), and a uterine manipulator (216). While not shown, robotic surgical system (210) may include a support structure similar to support structure (12) and platform (14) of robotic surgical system (10).

Uterine manipulator (216) includes an assembly of first and second components that are completely separable from each other. Particularly, the first component is shown as a first shaft assembly (218), and the second component is shown as a tool drive adapter (220). While uterine manipulator (216) of the present example includes two components that are completely separable from one another, other variations may provide more than two components that are completely separable from each other. While the present variation of surgical instrument (22, 114) is in the form of uterine manipulator (216), the following teachings may be readily applied to other variations of surgical instrument (22, 114). For instance, another variation of surgical instrument (22, 114) to which the following teachings may be applied may include a multi-part suction irrigator or any other suitable multi-part surgical instrument.

As will be described in greater detail below, uterine manipulator (216) has a two-part architecture that separates first shaft assembly (218) from tool drive adapter (220) during the process of insertion into the patient, resulting in significantly less mass at the proximal end of first shaft assembly (218) before docking of tool drive adapter (220) with robotic arm (212). First shaft assembly (218) is inserted through the vaginal canal into the uterus. First shaft assembly (218) is used to control the position of the uterus during gynecological procedures (e.g., during the colpotomy step of a hysterectomy). By way of example only, at least part of first shaft assembly (218) may be configured and operable in accordance with at least some of the teachings of U.S. patent application Ser. No. 17/468,754, filed Sep. 8, 2021, the disclosure of which is incorporated by reference herein, in its entirety.

With continued reference to FIGS. 4A-4B, robotic arm (212) includes proximal and distal ends (222, 224). Robotic arm (212) includes a plurality of links (226) similar to links (116). Links (226) are connected at pivotable joints (228) similar to joints (118). While two links (226) and two joints (228) are shown in FIGS. 4A-4B, additional links (226) and joints (228) are envisioned to provide greater freedom of movement (e.g., four, five, six, or more links (226) and joints (228) are envisioned). Joints (228) are configured to provide a range of movements similar to robotic arms (16, 110) of FIGS. 1-2. Proximal end (222) of robotic arm (212) may be operatively coupled with support structure (12) of FIG. 1. For example, proximal end (222) of robotic arm (212) may be operatively coupled with base (18) and/or column (20) (e.g., via carriage (26)). Robotic arm (212) is configured to position uterine manipulator (216) at the desired position relative to the patient (P). Particularly, robotic arm (212) may move tool drive assembly (214) and uterine manipulator (216) relative to the patient (P) as instructed by control console (28) of FIG. 1. Additional robotic arms (212) are also envisioned (see FIG. 1). For example, another robotic arm (212) may be configured to support one or more additional surgical instruments (22, 114) (e.g., a laparoscope, cutting instrument, stapler, etc.).

Tool drive assembly (214) is operatively coupled with distal end (224) of robotic arm (212). Tool drive assembly (214) includes a stage (230), a carriage (232), and a coupling feature (234). Tool drive assembly (214) of the present example further includes a disengagement feature (236) and control features (238, 240), though one or more of these features (236, 238, 240) may be omitted in some versions. Stage (230) includes first and second tracks (242a-b), though some variations may include just one track (242) or more than two tracks (242). First and second tracks (242a-b) extend generally parallel to each other. Tool drive assembly (214) includes proximal and distal ends (244, 246). Coupling feature (234) is positioned at distal end (246) of tool drive assembly (214), and is described in additional detail below with reference to FIG. 8. In some versions, coupling feature (234) of tool drive assembly (214) may be referred to as a stage cannula mount. Control features (238, 240) may control one or more operations associated with carriage (232). For example, control features (238, 240) may include one or more single function or multi-function buttons to control the movement of carriage (232).

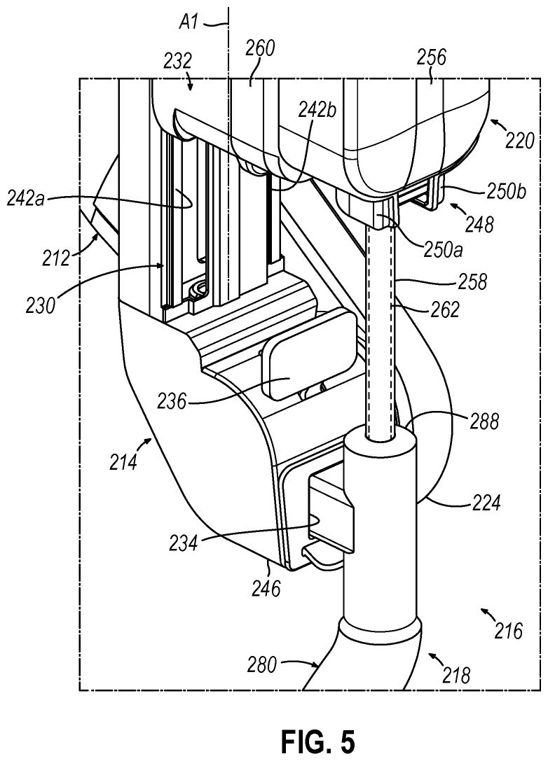

Carriage (232) is configured to move relative to stage (230). Particularly, carriage (232) is configured to move tool drive adapter (220) from a non-engaged position as shown in FIG. 4A to an engaged position as shown in FIG. 4B. For example, carriage (232) may translate along first and second tracks (242a-b) between the non-engaged configuration and the engaged configuration. FIG. 4A shows a non-coupled and non-engaged configuration. In other words, FIG. 4A shows robotic surgical system (210) prior to first shaft assembly (218) being coupled with tool drive assembly (214) to form the non-engaged configuration. In the non-engaged configuration, first shaft assembly (218) is spaced a distance from tool drive adapter (220). In the engaged configuration of FIG. 4B and FIG. 5, first shaft assembly (218) and tool drive adapter (220) are engaged. Carriage (232) moves relative to stage (230) along an axis (A1) defined by stage (230).

Carriage (232) of the present example includes a support structure (248) that is configured to support tool drive adapter (220). As shown, support structure (248) includes first and second arms (250a-b) that support one or more components of tool drive adapter (220). However, other support structures are also envisioned. In some versions, support structure (248) may be omitted entirely such that carriage (232) is coupled with tool drive adapter (220) and/or cartridge (260) using one or more coupling features (254). As shown in FIGS. 4A-4B, first shaft assembly (218) may be effectively rotated in three different ways, including: roll (about the z axis), pitch (about the x axis), and yaw (about the y axis). Robotic arm (212) may drive rotation of the entire first shaft assembly (218) in any of these directions. In some versions, carriage (232) is operable to drive roll rotation of at least a portion of first shaft assembly (218). Carriage (232) may include outputs similar to rotary drives (136a-f) shown in FIG. 3B to control motion that is transferred to corresponding components of first shaft assembly (218). Carriage (232) may also translate along stage (230) to drive translation of at least part of first shaft assembly (218).

Tool drive adapter (220) is operatively coupled with tool drive assembly (214) in both the arrangement shown in FIG. 4A and the arrangement shown in FIG. 4B. Tool drive adapter (220) includes a handle (256), a second shaft assembly (258), and a cartridge (260). In some variations, the features of cartridge (260) are integrated into handle (256) and/or carriage (232), rather than cartridge (260) being provided as an additional component. Handle (256) is operatively coupled with carriage (232) of tool drive assembly (214) in both the arrangement shown in FIG. 4A and the arrangement shown in FIG. 4B. Second shaft assembly (258) extends distally from handle (256) and includes a tube (262). Some variations of second shaft assembly (258) may include other components, such as additional tubes, shafts, wires, illumination fibers, etc. Cartridge (260) of the present example is disposed between handle (256) and carriage (232). Cartridge (260) is configured to provide a sterile barrier between handle (256) and carriage (232). In some variations, the features of cartridge (260) are integrated into handle (256) and/or carriage (232), or cartridge (260) is otherwise omitted, such that cartridge (260) need not necessarily be provided as an additional component.

As shown in FIGS. 4A-4B, a pressurized fluid source (264) containing fluid is fluidly coupled with tool drive adapter (220). Similarly, a power source (266) may be electrically coupled with tool drive adapter (220) so as to allow power to be transferred between second shaft assembly (258) and first shaft assembly (218). Power source (266) may be used to provide power to an illuminating element (e.g., one or more LEDs, a lens illuminated by one or more optical fibers, etc.). In such versions, one or more wires, optical fibers, and/or other components may extend along the length of shaft assemblies (218, 258) to couple with a source of electrical power, a source of light, etc. In some versions, conduits for pressurized fluid source (264) and/or power source (266) may extend through robotic arm (212) and tool drive assembly (214).

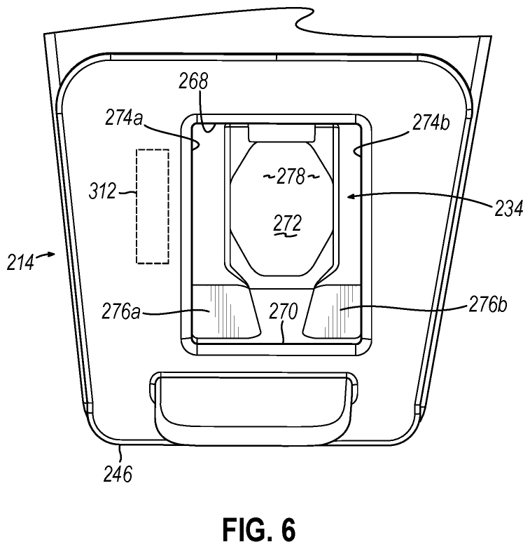

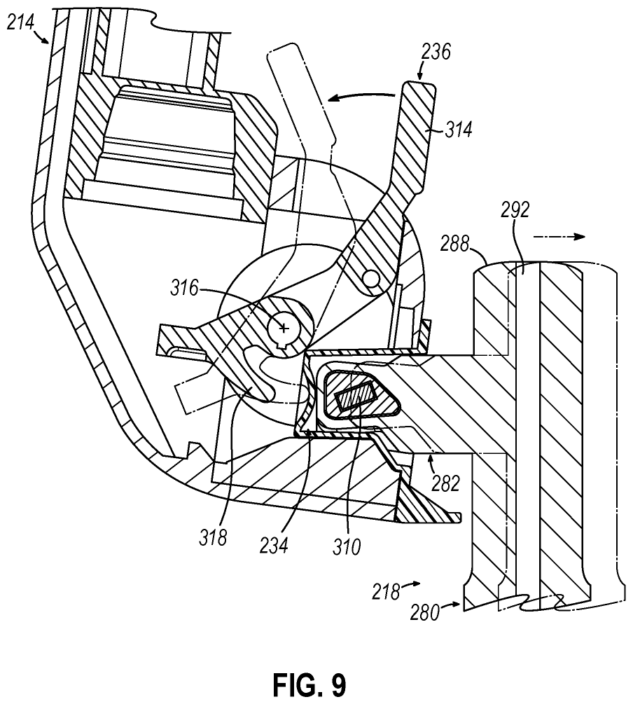

FIG. 6 shows an enlarged view of the coupling feature (234) of tool drive assembly (214). Coupling feature (234) includes an upper surface (268), a lower surface (270), a rear surface (272), and first and second side surfaces (274a-b). A first ramp (276a) extends from first side surface (274a). Similarly, a second ramp (276b) extends from second side surface (274b). First and second ramps (276a-b) are separated by a cavity (278). First and second ramps (276a-b) are configured to guide coupling feature (282) of first shaft assembly (218) into position as shown in the cross-sectional view of FIG. 9.

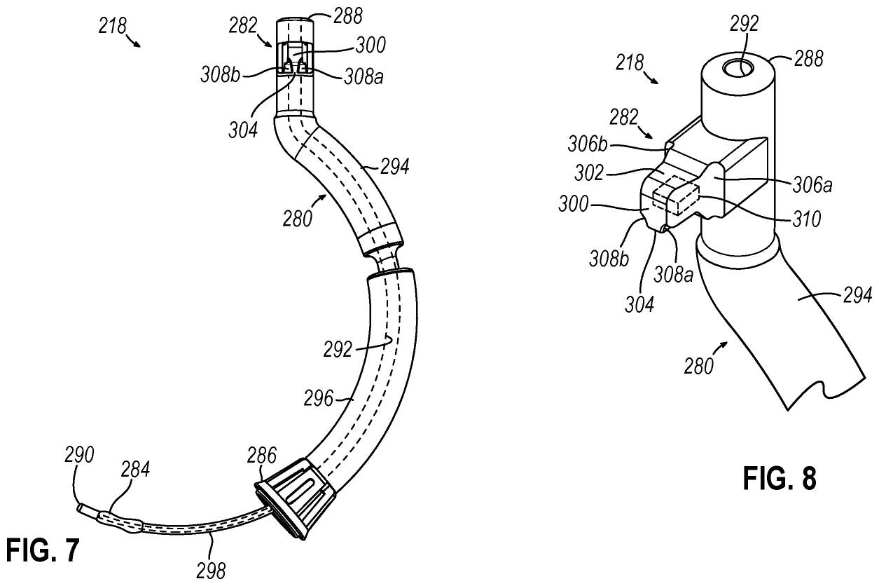

FIGS. 4A-4B and FIGS. 7-8 show first shaft assembly (218), a distal portion of which is configured to be inserted into the uterus of the patient (P). As shown in FIG. 7, first shaft assembly (218) includes a shaft subassembly (280), a coupling feature (282), an expandable balloon (284), a colpotomy cup (286), a proximal end (288), and a distal end (290). Shaft subassembly (280) includes one or more lumens (292) extending at least partially therethrough. Shaft subassembly (280) includes a proximal portion (294), a translatable sleeve (296), and a transcervical shaft (298). Sleeve (296) is slidably coupled to an outer surface of transcervical shaft (298), such that sleeve (296) may slide along transcervical shaft (298) from a proximal position (FIG. 7) to any number of distal positions. Proximal portion (294) may be rigid or substantially rigid. Sleeve (296) is generally cylindraceous and rigid; and extends along a curved axis such that the curved lateral profile complements the curved lateral profile of transcervical shaft (298). Sleeve (296) may be formed of plastic, metal, and/or any other suitable biocompatible material(s), including combinations of materials. Expandable balloon (284) is positioned at distal end (290) of a transcervical shaft (298). The interior of sleeve (330) includes lumen (292) that is configured to communicate pressurized fluid from pressurized fluid source (264) to expandable balloon (284). Lumen (292) may be in fluid communication with expandable balloon (290) via a lumen formed through transcervical shaft (298).

Expandable balloon (284) is configured to transition from a non-inflated configuration as show in FIG. 4A to an inflated configuration as shown in FIG. 4B by transferring fluid from pressurized fluid source (264) through tube (262), lumen (292), and transcervical shaft (298) into expandable balloon (284). Expandable balloon (284) may be formed of an extensible material or a non-extensible material. Colpotomy cup (286) is slidably attached along a length of shaft subassembly (280). First shaft assembly (218) may include additional aspects and may function similar to robotic uterine manipulators shown and described in U.S. patent application Ser. No. 17/468,754 filed Sep. 8, 2021, the disclosure of which is incorporated by reference herein, in its entirety.

FIG. 8 shows an enlarged view of the coupling feature (282) of first shaft assembly (218). Coupling feature (282) of first shaft assembly (218) is disposed at proximal end (288) first shaft assembly (218). Coupling feature (282) extends generally perpendicular to shaft subassembly (280). As shown, coupling feature (282) includes a front surface (300), an upper tapered surface (302), a lower surface (304), and first and second outwardly tapering lateral surfaces (306a-b). Lower surface (304) separates first and second recessed surfaces (308a-b). During the coupling of coupling feature (282) of first shaft assembly (218) with coupling feature (234) of tool drive assembly (214), lower surface (304) is configured to contact lower surface (270) and be guided by sliding along first and second ramps (276a-b) (see FIG. 6).

FIG. 9 shows a cross-sectional view of coupling feature (282) of first shaft assembly (218) coupled with coupling feature (234) of tool drive assembly (214). While coupling feature (282) is shown as including a projection and coupling feature (234) is shown as including a corresponding cavity configured to receive the projection, this relationship may be reversed. For example, while not shown, coupling feature (282) may include a cavity, and coupling feature (234) may include a projection. As shown, coupling feature (282) includes a magnet (310) configured to couple with a metallic feature (312) of coupling feature (234) of tool drive assembly (214).

FIG. 9 shows disengagement feature (236) being moved from a first position (shown in solid lines) to a second position (shown in broken lines). Disengagement feature (236) is shown as a lever that includes a user contact feature (314) that is configured to be actuated by a user (e.g., a clinician) to pivot disengagement feature (236) at a pivot point (316). In some versions, disengagement feature (236) may be configured to lock and/or unlock coupling feature (234) of tool drive assembly (214) relative to coupling feature (282) of first shaft assembly (218). Rotation of disengagement feature (236) pushes coupling feature (282) of first shaft assembly (218) away from coupling feature (234) of tool drive assembly (214). Particularly, a projection (318) of disengagement feature (236) may eject coupling feature (282).

B. Example of a Method of Use

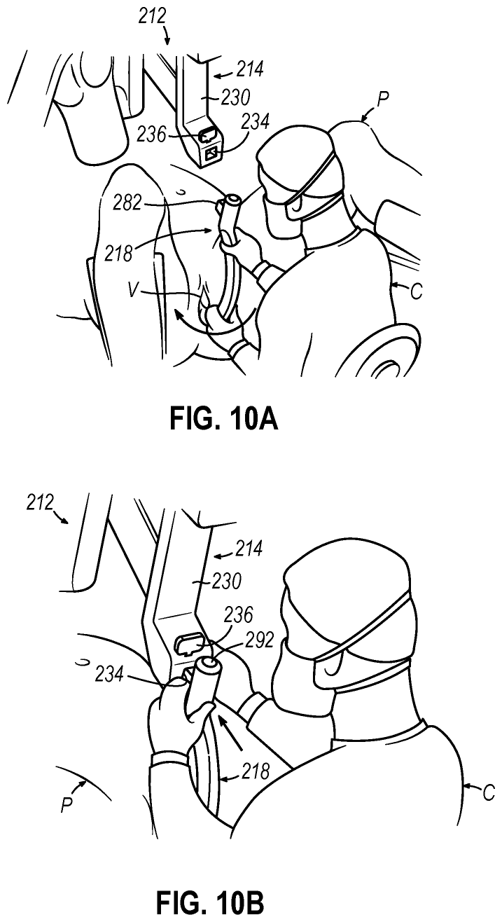

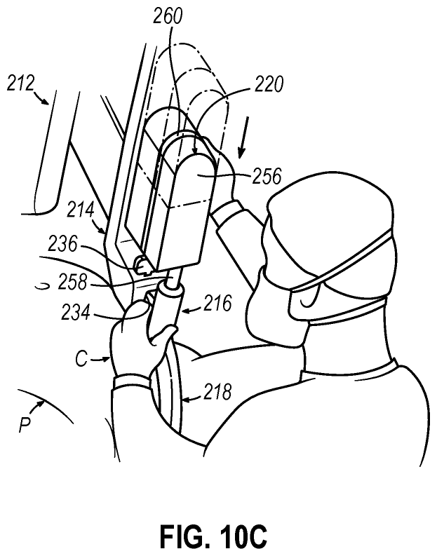

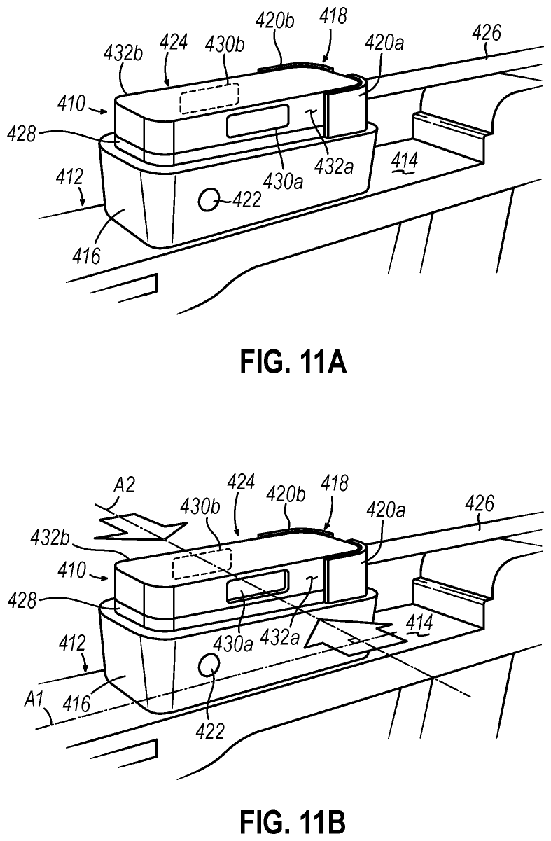

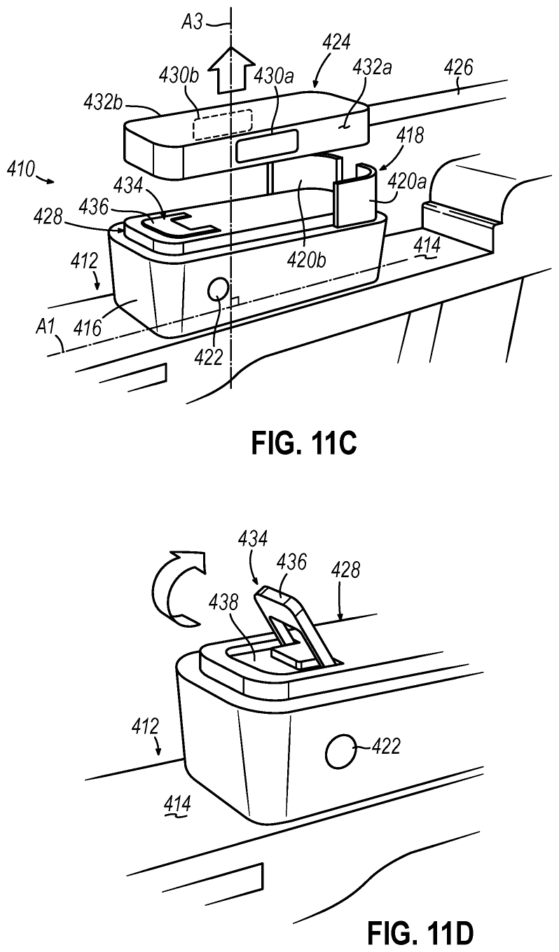

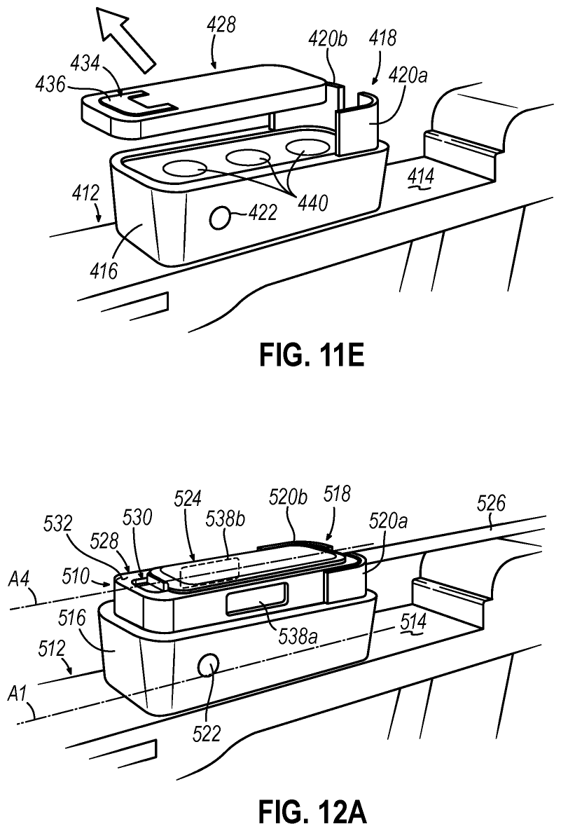

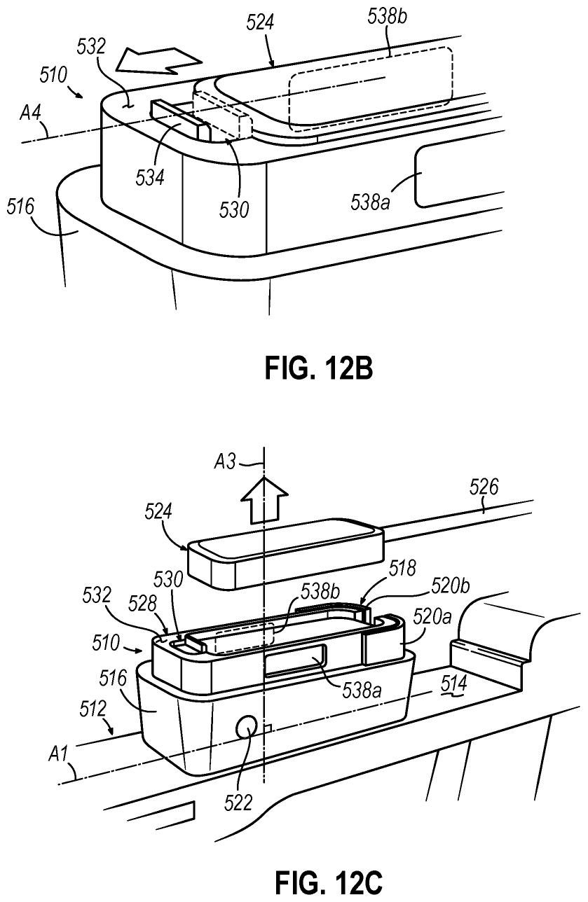

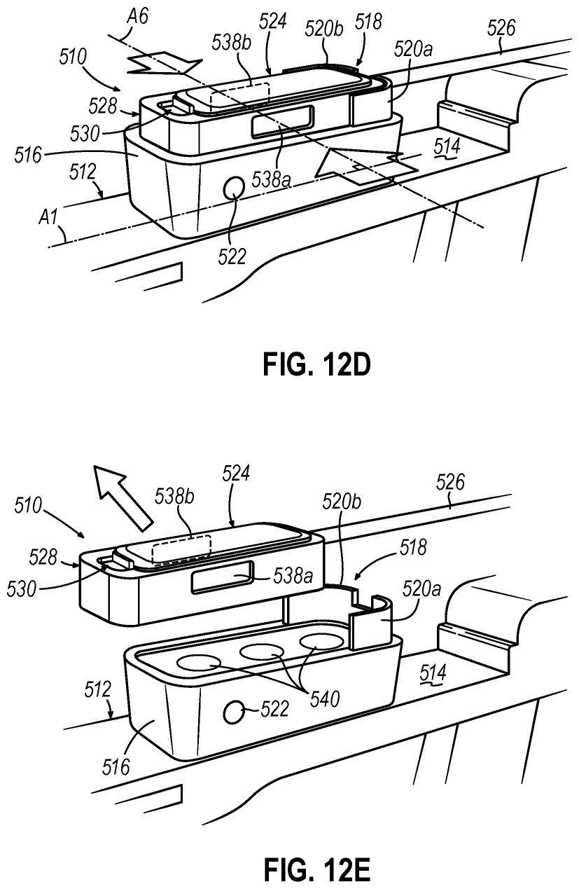

FIGS. 10A-10C show an example of a method of operating uterine manipulator (216). While not shown, the method may include operatively coupling tool drive adapter (220) of uterine manipulator (216) with carriage (232) of tool drive assembly (214). For example, tool drive adapter (220) may be pre-loaded onto carriage (232) prior to the start of the surgical procedure to reduce the amount of time first shaft assembly (218) remains free and unsupported in the patient (P). However, it is also envisioned that drive adapter (220) may be coupled carriage (232) after first shaft assembly (218) has been positioned within the patient (P). As will be described in FIGS. 11A-11E and 12A-12E with respect to handles (424, 524) and cartridges (428, 528), handle (256) may optionally include a coupling feature configured to couple with cartridge (260), and cartridge (260) may include a corresponding coupling feature configured to couple with carriage (232). In some versions, cartridge (260) may be omitted so that handle (256) directly couples with carriage (232).

As shown in FIG. 10A, the method includes manually inserting a distal end (290) of first shaft assembly (218) into the vagina (V) of the patient (P), to a point where transcervical shaft (298) traverses the cervix and expandable balloon (284) is disposed in the uterus. As shown, this is done before the proximal end of first shaft assembly (218) is coupled with tool drive adapter (220) or robotic arm (212), without the proximal end of first shaft assembly (218) being coupled with anything else, and without a substantially heavy mass positioned at the proximal end of first shaft assembly (218). After first shaft assembly (218) is inserted into the vagina (V), shaft assembly (218) may be left unsupported by the user (e.g., clinician). In other words, the first shaft assembly (218) may be positioned within the patient (P) without the weight of tool drive adapter (220), or other features that might otherwise act upon the proximal end of first shaft assembly (218), impeding control of first shaft assembly (218) by the clinician. This may provide an enhanced patient experience.

Conversely, in some other scenarios where first shaft assembly (218) is already coupled with tool drive adapter (220) before distal end (290) is inserted into the patient (P), the weight of tool drive adapter (220) bearing at the proximal end of first shaft assembly (218) may tend to cause an unbalanced and awkward experience for the user (e.g., the clinician) while inserting first shaft assembly (218) into the patient (P). This same situation may arise when using a one-part configuration that combines first shaft assembly (218) with tool drive adapter (220) is inserted into the patient. The mass of tool drive adapter (220) may tend to cause distal end (290) of first shaft assembly (218) to slip out of the patient (P) or otherwise move within the patent (P). It may be desirable to reduce or altogether eliminate movement of first shaft assembly (218) when distal end (290) is positioned within the patient (P), before tool drive adapter (220) is coupled with first shaft assembly (218).

As shown in FIG. 10B, the method includes coupling first shaft assembly (218) with tool drive assembly (214) of robotic surgical system (210). Particularly, coupling feature (282) of first shaft assembly (218) is coupled with coupling feature (234) of tool drive assembly (214) as shown and described in detail above with reference to FIGS. 4A-9. As part of this process, the user (e.g., clinician) may grasp tool drive assembly (214) and thereby manually manipulate robotic arm (212) to position tool drive assembly (214) adjacent to coupling feature (282) of first shaft assembly (218). The user (e.g., clinician) may then couple first shaft assembly (218) with tool drive assembly (214) using one hand or both hands while distal end (290) of first shaft assembly (218) remains within the vagina (V) of the patient (P). This coupling may be magnetically assisted using magnet (310) and metallic feature (312) as shown in FIG. 9.

As shown in FIG. 10C, the method of the present example further includes moving tool drive adapter (220) relative to first shaft assembly (218). As shown, carriage (232) and tool drive adapter (220) may be translated together along stage (230) of tool drive assembly (214) from the non-engaged configuration (shown using broken lines in FIG. 10C) to the engaged configuration (shown using solid lines in FIG. 10C). After moving tool drive adapter (220) relative to first shaft assembly (218), tool drive adapter (220) is coupled (e.g., “docked”) with first shaft assembly (218). Using the combination of first shaft assembly (218) and tool drive adapter (220) may tend to provide greater balance in the clinician's hand during insertion of first shaft assembly (218). This may also tend to allow first shaft assembly (218) to remain unsupported inside of the patient (P) before docking of first shaft assembly (218) with tool drive adapter (220).

The method of the present example also includes manipulating the uterus of the patient (P) using uterine manipulator (216) after uterine manipulator (216) is coupled with tool drive assembly (214). For example, movement of first shaft assembly (218) of uterine manipulator (216) may be controlled by movements in robotic arm (212) and/or movements by carriage (232) relative to stage (230). This movement may include translational movement and/or rotational movement (i.e., pitch, roll, and azimuth). Such manipulation of the uterus may be carried out in accordance with at least some of the teachings of U.S. patent application Ser. No. 17/468,754 filed Sep. 8, 2021, the disclosure of which is incorporated by reference herein, in its entirety.

At the end of the surgical procedure or when otherwise desired by the user, tool drive adapter (220) and cartridge (260) may be decoupled from carriage (232). For example, tool drive adapter (220) may be removed from cartridge by actuating a button, latch, and/or lever. Additional versions are shown and described below with reference to FIGS. 11B-11C and FIGS. 12B-12E. In some instances, it may be beneficial to have at least one of first shaft assembly (218) or tool drive adapter (220) immediately releasable from robotic surgical system (210). In some versions, tool drive adapter (220) may be raised from the position shown in FIG. 4B (or another example of a position) prior to or after first shaft assembly (218) is removed from tool drive assembly (214). This decoupling may be performed by removing coupling feature (282) of first shaft assembly (218) from coupling feature (234) of tool drive assembly (214). As described above, the user may manipulate disengagement feature (236) as part of the process of removing coupling feature (282) of first shaft assembly (218) from coupling feature (234) of tool drive assembly (214). Once coupling feature (282) has been disengaged from coupling feature (234), first shaft assembly (218) may be manually removed from the patient (P).

C. Example of a Method of Removing Tool Drive Adapter

FIGS. 11A-11E show an example of a method of removing a component of a surgical instrument. Particularly, FIGS. 11A-11E show a method of removing an example of a tool drive adapter (410) from a tool driver (412) using a sequenced de-latching. In this version, tool drive adapter (410) is similar to tool drive adapter (220), and tool driver (412) is similar to tool drive assembly (214), except as otherwise indicated below. Tool drive adapter (410) may be incorporated as part of a multi-part uterine manipulator, a multi-part suction irrigator, or another multi-part surgical instrument.

As shown in FIG. 11A, tool driver (412) includes a stage (414) similar to stage (230), and a carriage (416) similar to carriage (232). Similar to carriage (232), carriage (416) is configured to move relative to stage (414) along an axis (A1). While not shown, tool driver (412) may include a coupling feature similar to coupling feature (234). Similar to support structure (248), carriage (416) may include a support structure (418). As shown, support structure (418) includes first and second arms (420a-b) that support one or more components of tool drive adapter (410). Carriage (416) may also include at least one button (422) configured to control an operation of carriage (416).

With continued reference to FIG. 11A, tool drive adapter (410) includes a handle (424), a shaft assembly (426), and a cartridge (428). Shaft assembly (424) extends distally from handle (424). Handle (424) includes at least one coupling feature, shown as first and second coupling features (424a-b). First and second coupling features (430a-b) are disposed on first and second lateral sides (432a-b) of handle (424).

As shown in FIG. 11B, first and second coupling features (430a-b) are actuated along an axis (A2) that is perpendicular to axis (A1). In other words, the de-latching motion of handle (424) occurs in a direction that is perpendicular to that used for translation of carriage (416). As shown, first and second coupling features (430a-b) are depressed inwardly and mechanically unlock handle (424) from cartridge (428).

As shown in FIG. 11C, handle (424) is configured to be removed along an axis (A3) that is perpendicular to both axes (A1, A2). Once handle (424) is removed, a coupling feature (434) of cartridge (428) is exposed. As shown in FIGS. 11C-11D, coupling feature (434) of cartridge (260) is visible only after removing handle (256). Actuation of coupling feature (434) allows for cartridge (428) to be removed. As shown, coupling feature (434) includes a rotatable latch (436) that is configured to pivot distally. Rotatable latch (436) may be positioned within a recessed portion of cartridge (428), so as not to obstruct the connection between handle (424) and cartridge (428). As shown in FIG. 11D, rotatable latch (436) is rotated away from a body of cartridge to decouple cartridge from carriage (416). While a rotatable latch (436) is shown, other coupling features are also envisioned. Carriage (416) includes outputs (440) similar to rotary drives (136a-f) shown in FIG. 3B to control the motion that is transferred to first shaft assembly (218). While three outputs are shown, more or fewer outputs are also envisioned.

D. Examples of Alternative Methods of Removing Tool Drive Adapter

FIGS. 12A-12E show two different methods of removing a portion of surgical instrument. Particularly, FIGS. 12A-12E show first and second decoupling sequences for removing an example of a tool drive adapter (510) from a tool driver (512). In this version, tool drive adapter (510) is similar to tool drive adapter (220), and tool driver (512) is similar to tool drive assembly (214) except as otherwise indicated. Tool drive adapter (510) may be incorporated as part of a two-part uterine manipulator, a two-part suction irrigator, or another multi-part surgical instrument. As shown in coupled configuration of FIG. 12A, tool driver (512) includes a stage (514) similar to stage (230), and a carriage (516) similar to carriage (232). Similar to carriage (232), carriage (516) is configured to move relative to stage (514) along axis (A1).

While not shown, tool driver (512) may include a coupling feature similar to coupling feature (234). Similar to support structure (248), carriage (516) may include a support structure (518). As shown, support structure (518) includes first and second arms (520a-b) that vertically support one or more components of tool drive adapter (510). Carriage (516) may also include at least one button (522) configured to control an operation of carriage (516).

In a first decoupling sequence shown in FIGS. 12B-12C, handle (524) is configured to be removed from cartridge (528) while cartridge is still attached to carriage (232). As shown in FIG. 12A, tool drive adapter (510) includes a handle (524), a shaft assembly (526), and a cartridge (528). Shaft assembly (526) extends distally from handle (524). Cartridge (528) includes a coupling feature (530) disposed on a front surface (532) of cartridge (528). As shown in FIG. 12B, coupling feature (530) is configured to be actuated along an axis (A4) that extends parallel to and is offset from axis (A1). As shown in FIG. 12C, handle (524) is configured to be removed along an axis (A5) that is perpendicular to both axes (A1, A4). In other words, the de-latching motion of handle (524) occurs in a direction that is perpendicular to the direction of translation of carriage (516). As shown, cartridge (528) may surround an outer perimeter of handle (524). As shown in FIGS. 12B-12C, a translatable latch (534) of coupling feature (530) is configured to be moved proximally within a recess (536) so as to mechanically release handle (524) from cartridge (528). While a translatable latch (534) is shown, other coupling features are also envisioned.

In a second decoupling sequence shown in FIGS. 12D-12E, handle (524) and cartridge are collectively removed as a unit from carriage (232). In other words, cartridge (528) is removed while remaining coupled with handle (524). As shown in FIG. 12D, first and second coupling features (534a-b) of cartridge (528) are actuated along an axis (A6) that is perpendicular to axis (A1). As shown, first and second coupling features (534a-b) are depressed inwardly and mechanically unlock cartridge (528) from handle (524) from carriage (516). For example, the clinician (C) may use a pinching action to actuate first and second coupling features (534a-b). As a result, handle (524) may be removed from cartridge (528) while cartridge (528) is still attached to carriage (516). Carriage (516) includes outputs (540) similar to rotary drives (136a-f) shown in FIG. 3B to control the motion that is transferred to first shaft assembly (218). While three outputs are shown, more or fewer outputs are also envisioned.

### III. Examples of Combinations

The following examples relate to various non-exhaustive ways in which the teachings herein may be combined or applied. The following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A robotic surgical system, comprising: (a) a robotic arm that includes a distal end; (b) a tool driver operatively coupled with the distal end of the robotic arm; and (c) a surgical instrument comprising: (i) a uterine manipulator configured to be inserted into a uterus of a patient, and (ii) a tool drive adapter configured to move from a non-engaged configuration where the tool drive adapter is spaced a distance from the uterine manipulator to an engaged configuration where the uterine manipulator and the tool drive adapter are engaged, wherein the tool drive adapter is operatively coupled with the tool driver in the non-engaged and engaged configurations.

### Example 2

The robotic surgical system of Example 1, wherein the uterine manipulator includes a coupling feature, wherein the tool driver includes a coupling feature configured to couple with the coupling feature of the first component.

### Example 3

The robotic surgical system of Example 2, wherein the tool driver includes proximal and distal ends, wherein the coupling feature of the tool driver is positioned at the distal end of the tool driver.

### Example 4

The robotic surgical system of any of Examples 2 through 3, wherein the tool driver includes a decoupling feature configured to eject the coupling feature of the uterine manipulator from the coupling feature of the tool driver relative.

### Example 5

The robotic surgical system of any of Examples 1 through 4, the tool driver comprising: (i) a stage, and (ii) a carriage configured to move relative to the stage, wherein the carriage is configured to move the tool drive adapter from the non-engaged configuration to the engaged configuration.

### Example 6

The robotic surgical system of Example 5, wherein the stage includes at least one track, wherein the carriage is configured to translate along the at least one track between the non-engaged configuration and the engaged configuration.

### Example 7

The robotic surgical system of any of Examples 1 through 6, wherein the carriage is configured to move relative to the stage along a first axis, wherein the handle includes a coupling feature configured to be actuated along a second axis, wherein the second axis is perpendicular to the first axis.

### Example 8

The robotic surgical system of any of Examples 6 through 7, wherein the handle is configured to be removed along a third axis that is perpendicular to both the first and second axes.

### Example 9

The robotic surgical system of any of Examples 5 through 8, wherein the tool drive adapter includes a handle operatively coupled with the carriage of the tool driver in the non-engaged configuration and the engaged configuration.

### Example 10

The robotic surgical system of any of Examples 5 through 9, wherein the tool driver includes a cartridge positioned between the carriage and the handle.

### Example 11

The robotic surgical system of Example 10, wherein the cartridge includes a coupling feature that is visible only after removing the handle.

### Example 12

The robotic surgical system of any of Examples 10 through 11, wherein the carriage is configured to move relative to the stage along a first axis, wherein the cartridge includes a coupling feature that is configured to be actuated along a second axis that extends parallel to and is offset from the first axis.

### Example 13

The robotic surgical system of any of Examples 10 through 12, wherein the cartridge is configured to be removed from the carriage with the handle remaining coupled with the cartridge in a first decoupling sequence, wherein the handle is configured to be removed from the cartridge while the cartridge is still attached to the cartridge in a second decoupling sequence.

### Example 14

The robotic surgical system of any of Examples 10 through 13, wherein the uterine manipulator is completely separate from the tool drive adapter in the non-engaged configuration.

### Example 15

The robotic surgical system of any of Examples 1 through 14, further comprising a pressurized fluid source containing fluid, the uterine manipulator further comprising: (A) a shaft assembly that a lumen extending at least partially therethrough, and (B) an expandable balloon configured to be inflated by transferring fluid from the pressurized fluid source through the lumen and into the expandable balloon.

### Example 16

A robotic surgical system, comprising: (a) a robotic arm; (b) a tool driver operatively coupled with the robotic arm, the tool driver comprising: (i) a stage, and (ii) a carriage configured to move relative to the stage; and (c) a surgical instrument comprising: (i) a first component configured to be inserted into a patient, and (ii) a second component configured to move using the carriage from a non-engaged configuration where the first component is spaced a distance from the second component to an engaged configuration where the first and second components are engaged.

### Example 17

The robotic surgical system of Example 16, wherein the first component includes a uterine manipulator configured to be inserted into a uterus of the patient, wherein the second component includes a tool drive adaptor configured to couple with the uterine manipulator.

### Example 18

A method of operating a uterine manipulator assembly of a robotic surgical system, the method comprising: (a) manually inserting a distal end of a uterine manipulator of the uterine manipulator assembly into a patient; (b) coupling the uterine manipulator with a tool driver of the robotic surgical system after the distal end of the uterine manipulator is manually inserted into the patient; (c) moving a tool drive adapter of the uterine manipulator assembly relative to the uterine manipulator; (d) coupling the uterine manipulator and the tool drive adapter of the uterine manipulator assembly together; and (e) manipulating a uterus of the patient using the uterine manipulator assembly.

### Example 19

The method of Example 18, further comprising operatively coupling the tool drive adapter of the uterine manipulator assembly with a carriage.

### Example 20

The method of any of Examples 18 through 19, wherein moving the tool drive adapter relative to the uterine manipulator further comprises translating the tool drive adapter relative to the first component along a stage of the tool driver.

### IV. Miscellaneous

For clarity of disclosure, the terms “proximal” and “distal” are defined herein relative to a surgeon or other operator grasping a surgical instrument having a distal surgical end effector. The term “proximal” refers the position of an element closer to the surgeon or other operator and the term “distal” refers to the position of an element closer to the surgical end effector of the surgical instrument and further away from the surgeon or other operator.

It should be noted that the terms “couple,” “coupling,” “coupled” or other variations of the word couple as used herein may indicate either an indirect connection or a direct connection. For example, if a first component is “coupled” to a second component, the first component may be either indirectly connected to the second component via another component or directly connected to the second component.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

As used herein, the term “plurality” denotes two or more. For example, a plurality of components indicates two or more components.

It should be understood that any of the versions of the instruments described herein may include various other features in addition to or in lieu of those described above. By way of example only, any of the devices herein may also include one or more of the various features disclosed in any of the various references that are incorporated by reference herein. Various suitable ways in which such teachings may be combined will be apparent to those skilled in the art.

While the examples herein are described mainly in the context of uterine manipulator instruments, it should be understood that various teachings herein may be readily applied to a variety of other types of devices. By way of example only, the various teachings herein may be readily applied to other types of surgical instruments including tissue graspers, tissue retrieval pouch deploying instruments, surgical staplers, surgical clip appliers, ultrasonic surgical instruments, etc. It should also be understood that the teachings herein may be readily applied to any of the instruments described in any of the references cited herein, such that the teachings herein may be readily combined with the teachings of any of the references cited herein in numerous ways. Other types of instruments into which the teachings herein may be incorporated will be apparent to those skilled in the art.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those skilled in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions or other disclosure material set forth herein will only be incorporated to the extent that no conflict arises between that incorporated material and the existing disclosure material.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A robotic surgical system, comprising:
(a) a robotic arm that includes a distal end;
(b) a tool driver operatively coupled with the distal end of the robotic arm; and
(c) a surgical instrument comprising:
(i) a uterine manipulator configured to be inserted into a uterus of a patient, and
(ii) a tool drive adapter configured to move from a non-engaged configuration where the tool drive adapter is spaced a distance from the uterine manipulator to an engaged configuration where the uterine manipulator and the tool drive adapter are engaged, wherein the tool drive adapter is operatively coupled with the tool driver in the non-engaged and engaged configurations.

2. The robotic surgical system of claim 1, wherein the uterine manipulator includes a coupling feature, wherein the tool driver includes a coupling feature configured to couple with the coupling feature of the first component.

3. The robotic surgical system of claim 2, wherein the tool driver includes proximal and distal ends, wherein the coupling feature of the tool driver is positioned at the distal end of the tool driver.

4. The robotic surgical system of claim 2, wherein the tool driver includes a decoupling feature configured to eject the coupling feature of the uterine manipulator from the coupling feature of the tool driver relative.

5. The robotic surgical system of claim 1, the tool driver comprising:
(i) a stage, and
(ii) a carriage configured to move relative to the stage, wherein the carriage is configured to move the tool drive adapter from the non-engaged configuration to the engaged configuration.

6. The robotic surgical system of claim 5, wherein the stage includes at least one track, wherein the carriage is configured to translate along the at least one track between the non-engaged configuration and the engaged configuration.

7. The robotic surgical system of claim 6, wherein the carriage is configured to move relative to the stage along a first axis, wherein the handle includes a coupling feature configured to be actuated along a second axis, wherein the second axis is perpendicular to the first axis.

8. The robotic surgical system of claim 6, wherein the handle is configured to be removed along a third axis that is perpendicular to both the first and second axes.

9. The robotic surgical system of claim 5, wherein the tool drive adapter includes a handle operatively coupled with the carriage of the tool driver in the non-engaged configuration and the engaged configuration.

10. The robotic surgical system of claim 5, wherein the tool driver includes a cartridge positioned between the carriage and the handle.

11. The robotic surgical system of claim 10, wherein the cartridge includes a coupling feature that is visible only after removing the handle.

12. The robotic surgical system of claim 10, wherein the carriage is configured to move relative to the stage along a first axis, wherein the cartridge includes a coupling feature that is configured to be actuated along a second axis that extends parallel to and is offset from the first axis.

13. The robotic surgical system of claim 10, wherein the cartridge is configured to be removed from the carriage with the handle remaining coupled with the cartridge in a first decoupling sequence, wherein the handle is configured to be removed from the cartridge while the cartridge is still attached to the cartridge in a second decoupling sequence.

14. The robotic surgical system of claim 1, wherein the uterine manipulator is completely separate from the tool drive adapter in the non-engaged configuration.

15. The robotic surgical system of claim 1, further comprising a pressurized fluid source containing fluid, the uterine manipulator further comprising:
(A) a shaft assembly that a lumen extending at least partially therethrough, and
(B) an expandable balloon configured to be inflated by transferring fluid from the pressurized fluid source through the lumen and into the expandable balloon.

16. A robotic surgical system, comprising:
(a) a robotic arm;
(b) a tool driver operatively coupled with the robotic arm, the tool driver comprising:
(i) a stage, and
(ii) a carriage configured to move relative to the stage; and
(c) a surgical instrument comprising:
(i) a first component configured to be inserted into a patient, and
(ii) a second component configured to move using the carriage from a non-engaged configuration where the first component is spaced a distance from the second component to an engaged configuration where the first and second components are engaged.

17. The robotic surgical system of claim 16, wherein the first component includes a uterine manipulator configured to be inserted into a uterus of the patient, wherein the second component includes a tool drive adaptor configured to couple with the uterine manipulator

18. A method of operating a uterine manipulator assembly of a robotic surgical system, the method comprising:
(a) manually inserting a distal end of a uterine manipulator of the uterine manipulator assembly into a patient;
(b) coupling the uterine manipulator with a tool driver of the robotic surgical system after the distal end of the uterine manipulator is manually inserted into the patient;
(c) moving a tool drive adapter of the uterine manipulator assembly relative to the uterine manipulator;
(d) coupling the uterine manipulator and the tool drive adapter of the uterine manipulator assembly together; and
(e) manipulating a uterus of the patient using the uterine manipulator assembly.

19. The method of claim 18, further comprising operatively coupling the tool drive adapter of the uterine manipulator assembly with a carriage.

20. The method of claim 18, wherein moving the tool drive adapter relative to the uterine manipulator further comprises translating the tool drive adapter relative to the first component along a stage of the tool driver.
